(19) United States
(12) Patent Application Publication
(10) Pub. No.: US 2017/0189605 A1
(43) Pub. Date: Jul. 6, 2017

(54) MEDICAMENT INFUSION SYSTEM AND PUMP ASSEMBLY FOR USE THEREIN

(71) Applicant: SMITHS MEDICAL ASD ,INC., Plymouth, MN (US)

(72) Inventor: Michael L. BLOMQUIST, Plymouth, MN (US)

(73) Assignee: Smiths Medical ASD, Inc., Plymouth, MN (US)

(21) Appl. No.: 15/325,043

(22) PCT Filed: Jun. 29, 2015
(86) PCT No.: PCT/US2015/038309

§ 371 Date: Jan. 9, 2017

Related U.S. Application Data
(60) Provisional application No. 62/031,678, filed on Jul. 31, 2014.
(60) Provisional application No. 62/065,947, filed on Oct. 20, 2014.

(51) Int. Cl. A61M 5/142; A61M 39/10

(52) U.S. Cl. CPC A61M 5/142; A61M 39/10; A61M 2005/14208; A61M 2205/502

(57) ABSTRACT

An infusion system fully compliant with international standard ISO 80639-1, Small-bore Connectors for Liquids and Gases in Healthcare Applications, yet compatible with a multipurpose infusion pump to enable operational redeployment of the pump. An attachment set, removably couplable to the pump inlet and outlet, includes a coded attachment set outlet connector detachably coupling the attachment set to an infusion set, and a coded attachment set pump fitting coupling the attachment set to the infusion pump, thereby identifying to the pump a particular infusion route.

### TECHNICAL FIELD

Embodiments relate to medicament infusion systems. In particular, embodiments relate to a medicament infusion system incorporating a multipurpose, programmable pump assembly into a medicament infusion system, compliant with an approved connectivity standard. More particularly, embodiments provide for an infusion system that is fully compliant with international standard ISO 80639-1 (Small-bore Connectors for Liquids and Gases in Healthcare Applications; General Requirements), while enabling operational redeployment of a multipurpose infusion pump used in the infusion system.

### BACKGROUND

Infusion pumps are used to administer various types of drugs, nutritional compositions, and prescribed fluids or fluid-like substances (collectively, “medicaments”) to patients in volume and time controlled doses. The pumps can be used to transfer medicaments, that are stored in storage containers such as cassettes and bags, to be administered to patients via infusion systems through various routes of delivery, such as intravenously, neuraxially, and enterally. Of necessity, the infusion systems typically include various conduits and connectors for connecting the storage containers to the pumps, and the pumps to patients.

Luer connectors are commonly used to make leak-free connections between medicament containers, conduits, pumps and patients. A Luer male-taper fitting can quickly and effectively be inserted into a female part to effect a reliable fluid tight connection. Notwithstanding the effectiveness and ease of use provided by Luer connectors, concern has grown regarding the widespread use of a single type of connector in multiple applications that can be inherently incompatible. In particular, the use of a single type of connector invites the possibility of misconnecting a fluid source to an incompatible route of delivery. A medicament to be delivered enterally through a PEG tube, for example, could mistakenly be administered intravenously by misconnection to a peripheral cannula, if both the PEG tube and the cannula were fitted with the same type of connector. Even the same type of medicament will have different dosages depending on the route of delivery; and misapplication of either the medicament or the dosage through an inappropriate route of delivery can negate the curative benefit of the drug, and can, in some circumstances, even be fatal.

In an effort to reduce the potential for a misconnection leading to the introduction of a particular medicament via an undesired route of delivery or other error in dosage or administration, some users will mandate the use of certain pumps for certain routes of delivery or other such dedicated protocols in their health care facilities. For example, a particular brand and model of pump could be exclusively designated for use in neuraxial delivery applications. Users within a particular facility could be trained to recognize the particular brand and model of pump as being exclusively dedicated to the designated route, thereby reducing the chance of a wrong route administration for a particular patient. Such ad hoc efforts, however, do not provide the benefits of a universal standard; instead, these ad hoc efforts tend to artificially restrict the use of the health care facility's inventory of pumps while not necessarily restricting access as desired to improper delivery routes and the like.

ISO 80369, Small-bore Connectors for Liquids and Gases in Healthcare Applications (incorporated herein by reference in its entirety), is an emerging International Standard for connectivity between medical devices, patients, and accessories. Part 1 of ISO 80369, General Requirements (incorporated herein by reference in its entirety), was published in 2010, and Parts 2-7, addressing particular applications, are works in progress at the time of this disclosure. The ISO 80369 standard assigns specific connectors to specific routes of delivery, and makes those specific connectors exclusive to their designated route. Segregating medicaments by route of delivery, and designating unique connectors for the different routes of delivery, is intended to reduce the opportunity for administration of a particular medicine via an inappropriate route of delivery.

The primary routes of delivery for medicament infusion systems are intravenous (IV), neuraxial, and enteral. Examples of infusion pumps used in medicament infusion systems include so called ambulatory pumps such as those sold by an assignee of subject matter hereof under the trade names CADD™ Prizm, Cadd™ Legacy, and CADD™ Solis. Such pumps are multipurpose pumps in that each can be used with an IV, neuraxial or enteral route of delivery, as well as others, by simply programming the individual pump appropriately. It will be understood that although this disclosure refers to and presents examples of particular pumps, the subject matter hereof is applicable to any pump intended for administering medicaments such as syringe pumps, large volume pumps, elastomeric pumps and the like. On the other hand, ISO 80369, as a connectivity standard, segregates the connectors to be used for those three routes into separate categories, the connectors for each category being incompatible with, and unconnectable to, connectors from the other categories. While a multipurpose infusion pump can be used in different applications and for different delivery routes, the function of the pump needs to match, and needs to be restricted to, the delivery route it is assigned to, as do the connectors that incorporate the multipurpose pump into the infusion system, if the benefits of a connectivity standard are to be realized.

An infusion pump assembly that could incorporate a multipurpose infusion pump into an infusion system compliant with an established connectivity standard such as ISO 80369, without compromising the flexibility of use provided by the multipurpose infusion pump, would provide decided benefits.

### SUMMARY

The problems outlined above are in large measure addressed by embodiments of the present medicament infusion system. The medicament infusion system hereof includes a multipurpose, programmable infusion pump assembly. The infusion pump assembly comprises an infusion pump and a pump attachment set adapter for operably coupling the infusion pump into the medicament infusion system. The medicament infusion system includes standardized connectors keyed to a particular type of infusion route. The function of the infusion pump, for a particular deployment, is dictated by, and is exclusive to, the attachment set adapter, and the attachment set is in turn keyed to the type of standardized connectors, preferably ISO 80639 compliant connectors, being employed by the medicament infusion system. The pump can be redeployed for use with a differently configured infusion system with the change of the pump attachment set adapter. An attachment set adapter is exclusive to both the type of standardized connectors and the function of the pump, thereby coordinating proper operation of the pump with the type of standardized connectors and related infusion route.

The infusion pump, pump attachment set adapter and attachment set hereof comprise an infusion pump assembly for delivering a medicament from a medicament container, through an infusion set, to a patient, via an infusion route. The pump has a pump inlet and a pump outlet, and the pump attachment set adapter is removably couplable to the pump inlet and outlet. A coded pump attachment set adapter outlet connector operably couples the attachment set to an infusion set, for delivering medicament from the pump to a patient. The coded attachment set outlet connector, is preferably chosen from a group of ISO 80639 compliant connectors. The pump attachment set adapter includes a coded member communicatively couplable with the pump when the attachment set adapter is coupled to the pump. The coded member conveys information to the pump, identifying the type of standardized connectors employed in the medicament infusion system; and the pump is accordingly configured to operate in a manner compatible with the route of delivery associated with the standardized connectors.

A medicament infusion system hereof includes a pump with a pump inlet and pump outlet, and an attachment set that can be coupled to the pump inlet and outlet, the attachment set including an attachment set inlet connector operably, detachably coupling the attachment set to a medicament container, a coded attachment set outlet connector operably, detachably coupling the attachment set to an infusion set, the coded attachment set outlet connector being of a particular type selected from a group of different types of ISO 80369 compliant connectors, the coded attachment set outlet connector thereby identifying a particular infusion route, and a coded attachment set pump fitting in fluid communication with the attachment set inlet connector and the coded attachment set outlet connector, the coded attachment set pump fitting being operably, communicatively couplable with the pump to convey to the pump information regarding the coded attachment set outlet connector, thereby identifying to the pump the particular infusion route. The medicament infusion system can include a pump attachment set adapter operably, removably coupled to the pump for removably receiving the coded attachment set pump fitting, the pump attachment set adapter configured to operably, exclusively receive only a particular type of coded attachment set pump fitting selected from a plurality of differently coded types of attachment set pump fittings, whereby the operation of the pump is responsive to and exclusive to the coded attachment set outlet connector.

### BRIEF DESCRIPTION OF THE DRAWINGS

FIG. 1 is a block diagram depicting a medicament infusion system and pump assembly in accordance with an embodiment.

FIGS. 2a, 2b, and 2c each depict a medicament infusion pump, but with different attachment sets schematically and partially depicted, in accordance with embodiments.

FIG. 3 is a perspective view of an infusion pump adapted for use with the medicament infusion system in accordance with an embodiment, with the pump attachment set adapter removed.

FIG. 4 is an elevational view of a medicament infusion system and pump assembly in accordance with an embodiment.

FIG. 5 is a perspective, exploded view of a medicament infusion system and pump assembly in accordance with an embodiment, depicting an infusion pump and three variations of pump attachment set adapters and three sets of corresponding attachment sets, as detailed more particularly in the description of FIGS. 5a-g below.

FIGS. 5a, 5b, and 5c are partial perspective views depicting three variations of pump attachment set adapters in accordance with embodiments.

FIGS. 5d, 5e, and 5f are partial perspective views depicting three variations of attachment sets in accordance with embodiments, detailing the different coded pumping ports respectively corresponding with the pump attachment set adapters depicted in FIGS. 5a, 5b, and 5c.

FIG. 5g is a partial perspective view of an attachment set according to an embodiment, depicting the attachment set inlet connector and coded attachment set outlet connector.

FIGS. 6a, 6b, and 6c are enlarged, perspective views of the three variations of pump attachment set adapters depicted in FIGS. 5a, 5b, and 5c.

FIGS. 7a, 7b, and 7c are enlarged, perspective, partial views of the three variations of attachment sets depicted in FIGS. 5f, 5e, and 5d, respectively.

FIG. 8 is a partial, plan view of a medicament infusion system and pump assembly in accordance with an embodiment.

FIG. 9 is an enlarged, perspective view of a pumping port in accordance with an embodiment.

FIG. 10 is a partial, elevational view of a pump assembly with an alternative embodiment of an attachment set, the attachment set and pumping port aligned with but not coupled with the pump.

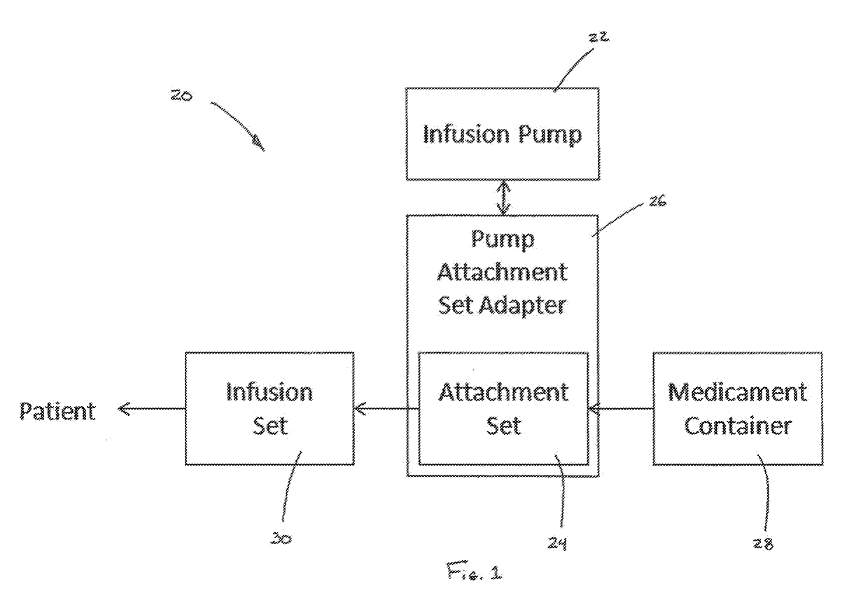

### DETAILED DESCRIPTION

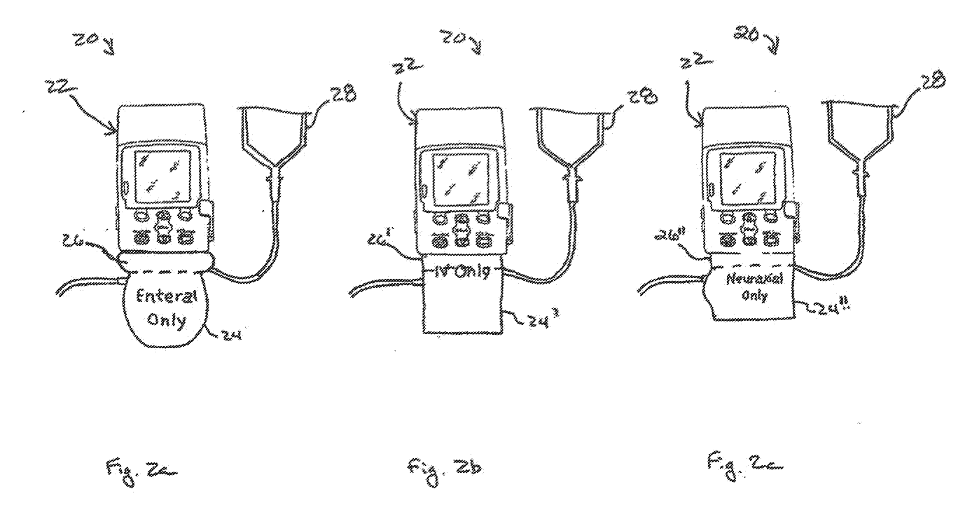

Referring to the drawings, a medicament infusion system 20 in accordance with an embodiment broadly includes a multipurpose infusion pump 22, an attachment set 24 and a pump attachment set adapter 26. Attachment set 24 is adapted for connection to a medicament container 28 and an infusion set 30. As schematically depicted in FIGS. 2a-2c, the infusion pump 22 can be made operative for pumping in a particular mode by changing the pump attachment set adapter 26 and attachment set 24. More particularly, with the use of different, interchangeable pump attachment set adapters 26, 26′ 26″ and corresponding attachment sets 24, 24′, 24″, multipurpose infusion pump 22 can be incorporated into an infusion system 20 that is fully compliant with an approved connectivity standard such as international standard ISO 80639-1 (Small-bore Connectors for Liquid and Gases in Healthcare Applications). Pump attachment set adapter 26 can comprise attachment set 24, or attachment set 24 can comprise pump attachment set adapter 26, or these devices can be considered to be distinct but cooperative, in various embodiments.

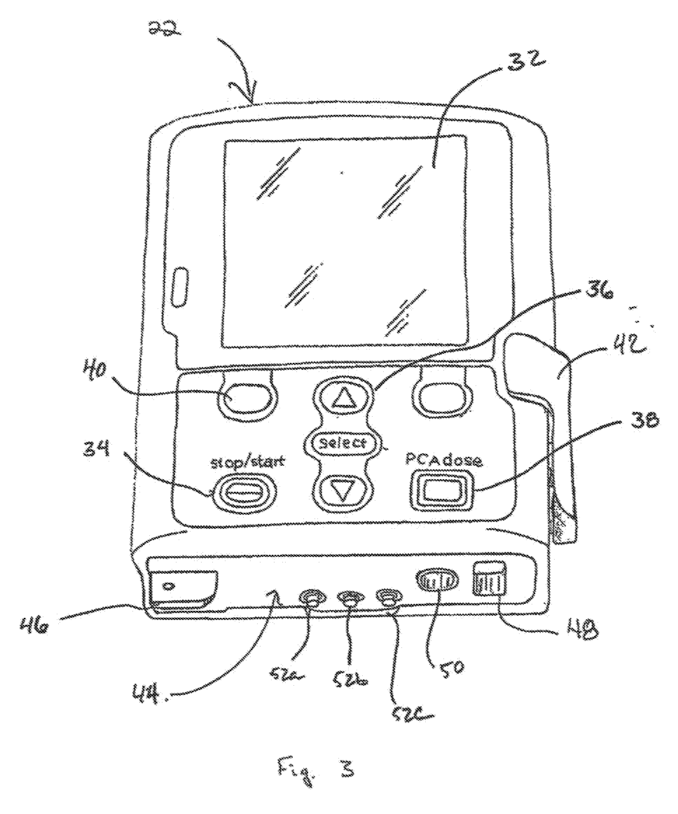

Referring to FIG. 3, infusion pump 22 can be a CADD®—Solis ambulatory infusion pump available from Smiths Medical, an assignee of the present application. The pump 22 includes a display screen 32, user interface buttons including stop/start button 34, scroll keys 36, PCA dose key 38, and soft key interface 40. Cassette latch 42 shifts between a latched position (shown) and release position (not shown). Operative connection of pump 22 with pump attachment set adapter 26 is facilitated along a bottom plate 44 of pump 22. Pump bottom plate 44 includes hinge fin 46, latch/lock port 48, inlet/outlet port 50 and detection pins 52a, 52b, 52c.

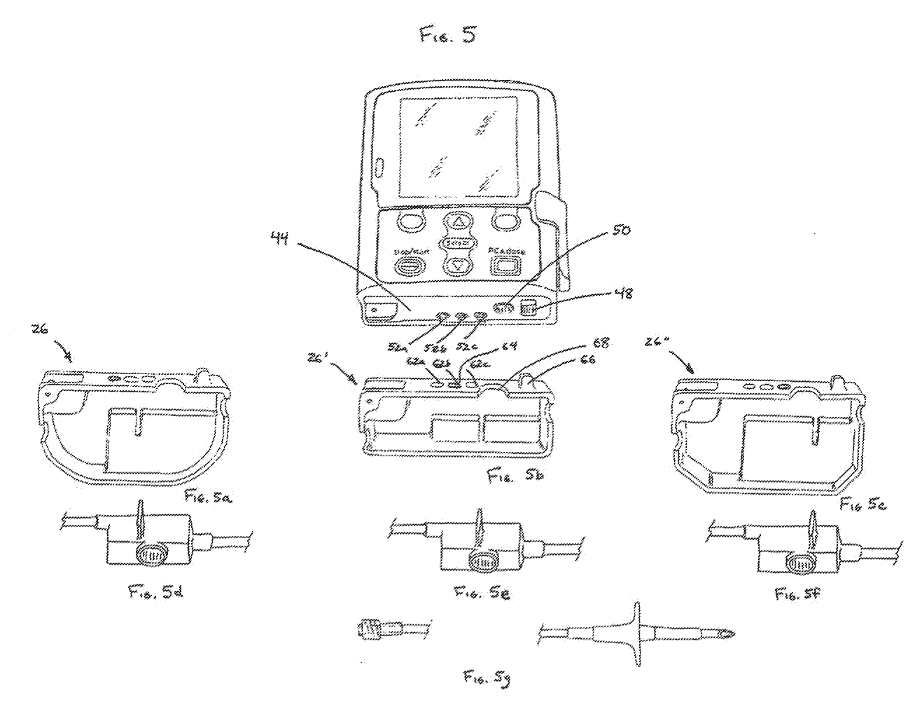

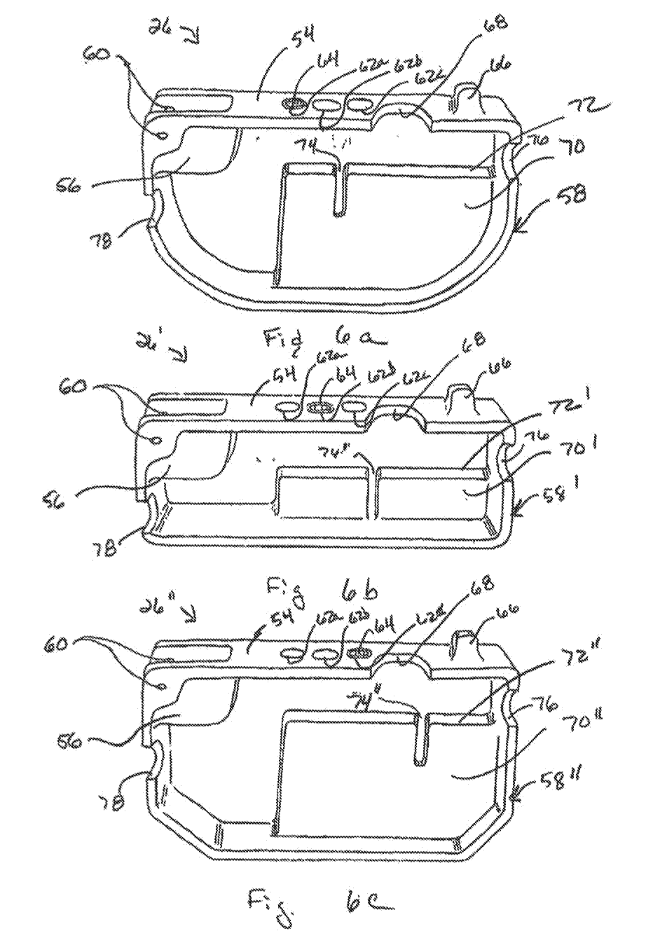

Referring to FIGS. 6a, 6b, and 6c, pump attachment set adapters 26, 26′, 26″ include pump interface top plate 54, pump hinge fin receptacle 56, and attachment set adapter body 58 (FIG. 6a), 58′ (FIG. 6b), 58″ (FIG. 6c). Pump hinge fin receptacle 56 includes hinge pin receiving apertures 60 for receiving an attachment hinge (not shown). Top plate 54 includes left, middle and right electrical contact receiving pads 62a, 62b, 62c, with a single electrical contact 64 carried in a different one of the pads 62a, 62b, 62c in the different attachment set adapter bodies 58, 58′, 58″, respectively, of FIGS. 6a, 6b, and 6c. Accordingly, the position of the single electrical contact 64 on a respective one of the pump attachment set adapters 26, 26′, 26″ signals to the infusion pump 22 the pump functionality (e.g., enteral, IV, or neuraxial) associated with the pump attachment set. It will be appreciated that the infusion pump 22, thereby receiving information as to pump configuration and functionality, can automatically adjust its drug library, its screen color, and warnings and alerts to be displayed, for example, according to the designated functionality of the pump. Top plate 54 further includes latch boss 66 and attachment set receiving notch 68. Referring to FIG. 5, it will be seen that the electrical contact receiving pads 62a, 62b, 62c, attachment set receiving notch 68, and latch boss 66 on the pump interface top plate 54 align with the detection pins 52a, 52b, 52c, inlet/outlet port 50 and latch lock port 48 on the pump bottom plate 44, respectively.

Referring to FIGS. 6a, 6b, and 6c, the different attachment set adapter bodies 58, 58′, 58″ intentionally differ in shape and size (depth) to be distinguishable from each other. Each of the bodies 58, 58′, 58″ includes a notch plate 70, 70′, 70″, respectively. Notwithstanding the intentionally different shapes and sizes of the attachment set adapter bodies 58, 58′, 58″, it will be seen that the top edge 72, 72′, 72″ of each of the notch plates 70, 70′, 70″, respectively, is approximately the same distance separation from the top plate 54 in each of the three variations. It will also be seen that the notch 74, 74′, 74″ for each respective notch plate 70, 70′, 70″ is in a different lateral orientation with respect to its respective attachment set receiving notch 68. Also, notwithstanding the different shapes and sizes of the attachment set adapter bodies, 58, 58′, 58″, each of the bodies includes an inlet notch 76 and outlet notch 78 that are respectively oriented in similar positions relative to their respective top plates 54, and in particular, relative to the attachment set receiving notch 68 of the top plate 54. The different shapes and sizes of the attachment set adapter bodies 58, 58′, 58″ enables visual distinction between different pump attachment set adapters 26, 26′, 26″, respectively.

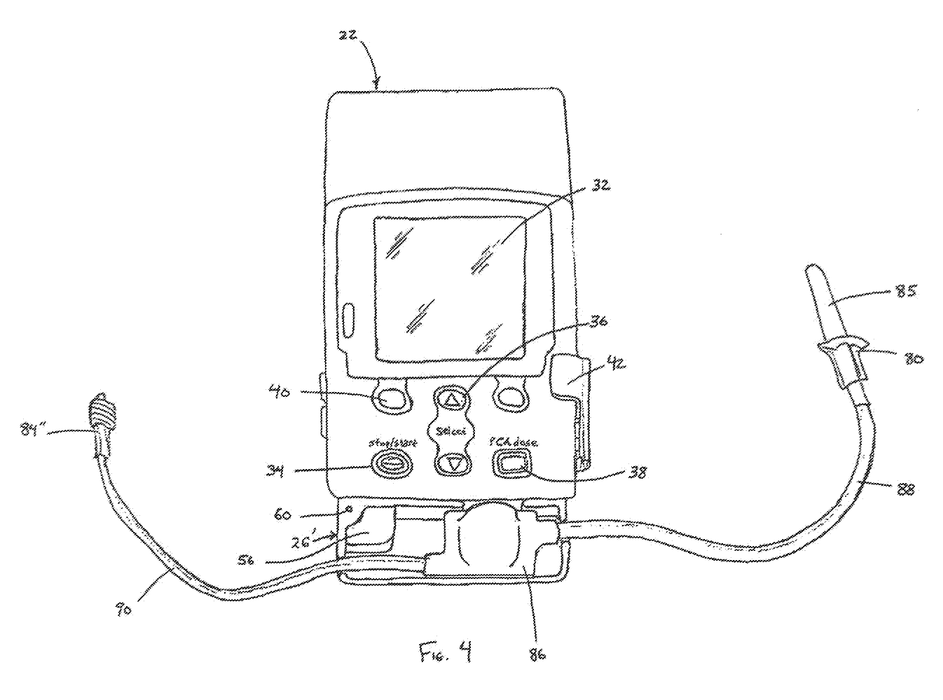

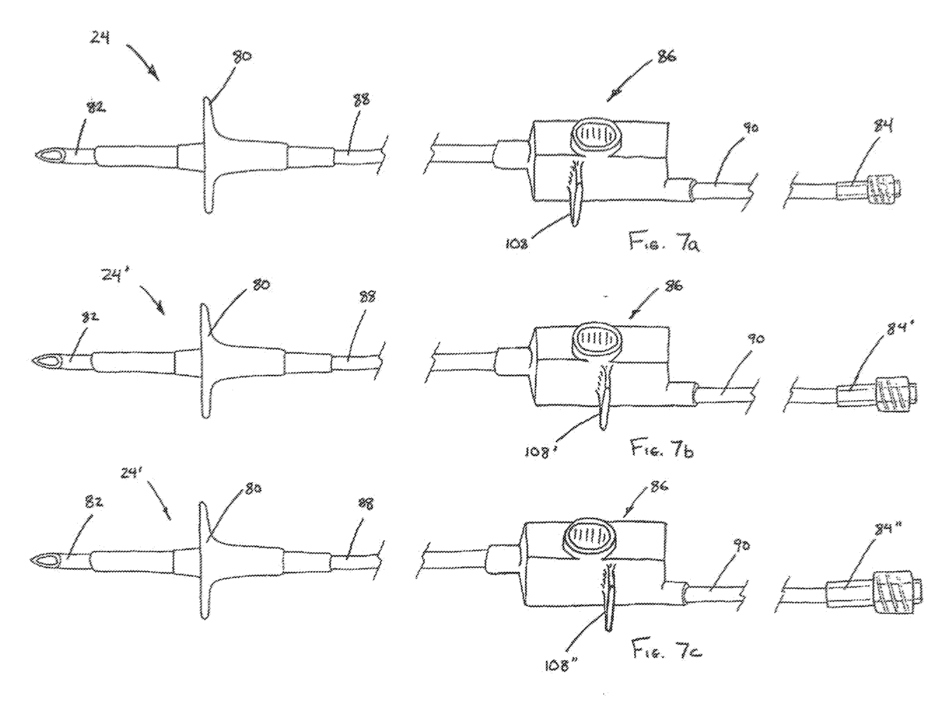

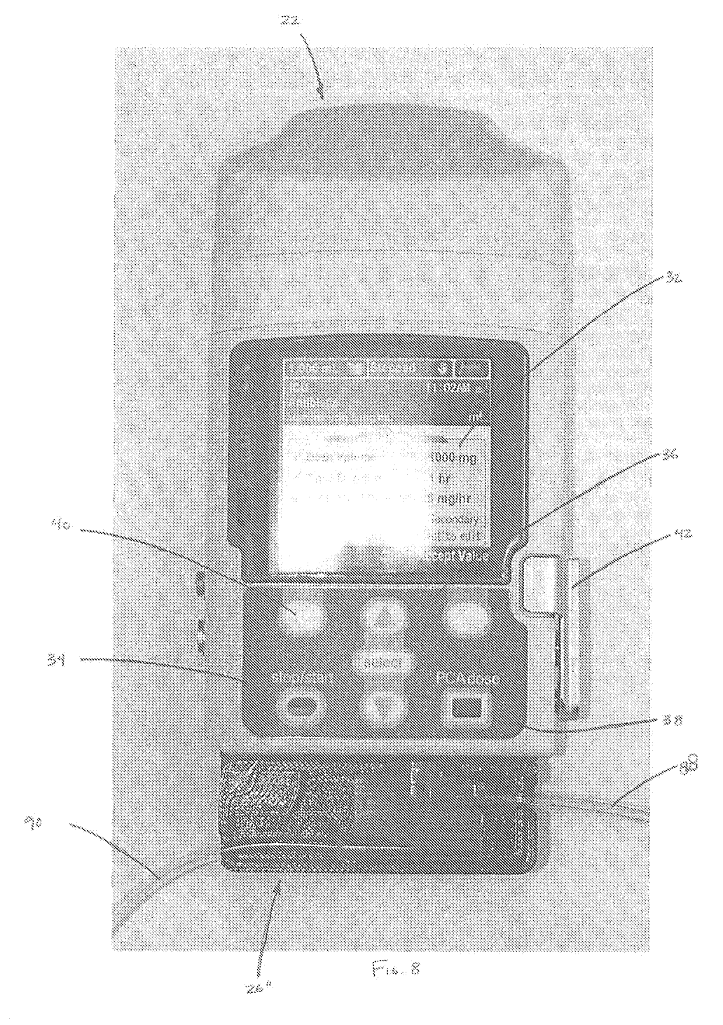

Referring to FIGS. 7a, 7b, and 7c, a respective attachment set 24, 24′, 24″ broadly includes an inlet connector 80, that includes a conventional sterile IV bag piercing spike 82, at one end, an outlet connector 84, 84′, 84″ for connecting each respective attachment set 24, 24′, 24″ to an infusion set 30 (not shown in FIGS. 7a-7c) at the opposite end, and an intermediate pumping port 86. Tubes 88 and 90 fluidly connect the inlet connector 80, pumping port 86 and respective outlet connectors 84, 84′, 84″. With reference to FIG. 4, bag piercing spike 82 can be provided with a cover 85.

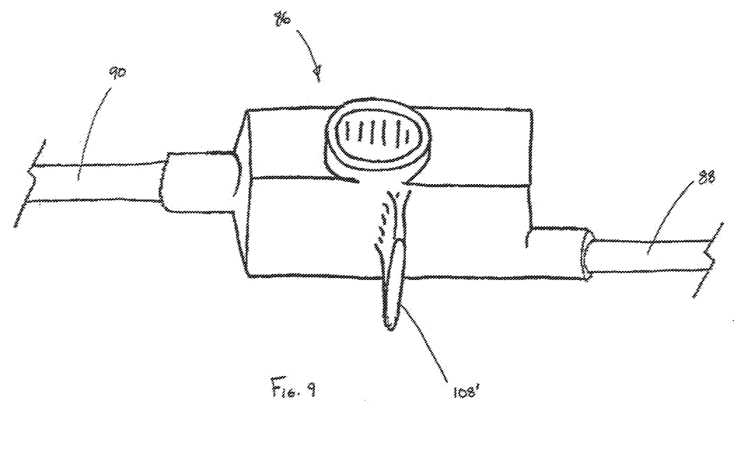

Referring to FIG. 9, pumping port 86 includes a guide fin 108, 108′, 108″ (108 and 108″ are not illustrated in FIG. 9) which each have a position on port 86 that is uniquely different corresponding to each of the three variations of the aforementioned attachment sets 24, 24′, 24″, respectively.

Referring again to FIGS. 7a, 7b, 7c, the respective outlet connectors 84, 84′, 84″ are uniquely different from each other, and further being a different one of a connector specified in ISO 80369, Small-bore Connectors for Liquids and Gases in Healthcare Applications. The positions of guide fins 108, 108′, 108″ on their respective ports 86 are coded to the particular type of outlet connector 84, 84′, 84″ of their respective attachment sets 24, 24′, 24″. In particular, by specifying that the positions of the guide fins 108, 108′, 108″ are to be uniquely and always associated with a particular respective outlet connector 84, 84′, 84″, the outlet connectors can be identified by the positions of their respective guide fins.

In addition, and again referring to FIGS. 6a, 6b, 6c, it will be seen that notch 74 (FIG. 6a), notch 74′ (FIG. 6b), and notch 74″ (FIG. 6c) of notch plates 70, 70′, 70″, respectively, are placed so as to only receive a complimentarily placed guide fin 108, 108′, 108″, respectively. Accordingly, it will be appreciated that the particular type of outlet connector 84, 84′, 84″ associated with a particularly placed guide fin 108, 108′, 108″ on its respective attachment set 24, 24′, 24″ as shown in FIGS. 7a-7c, can be uniquely identified to an infusion pump 22 by fitting the pump 22 with the appropriate attachment set adapter 26, 26′, 26″, that, as described, coordinates the placement of the notch 74, 74′, 74″ with the placement of the electrical contact 64, which placement conveys distinguishing information to the pump 22 by its location on the attachment set adapter 26, 26′, 26″.

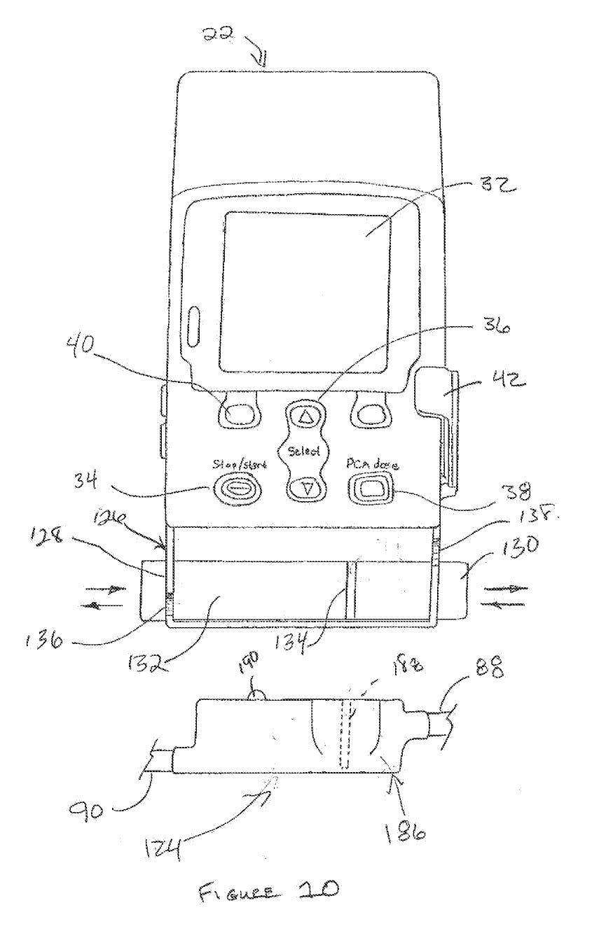

In an alternative embodiment, with reference to FIG. 10, the multipurpose infusion pump 22 includes alternative pump attachment set adapter 126 selectively couplable to alternative pumping port 186 of attachment set 124. Pump attachment set adapter 126 includes body 128 and shiftable key 130. Shiftable key 130 includes shiftable member 132 defining slot 134. Body 128 includes tube receiving notches 136, 138. Notch 136 can receive attachment set tube 90. Notch 138 can receive attachment set tube 88. Pumping port 186 includes guide fin 188 and indicator pin 190. In operation, with reference to FIGS. 2a-2c, the benefits of a multipurpose, programmable infusion pump 22 are incorporated into an infusion system 20 compliant with an established connectivity standard such as ISO 80369 by using the described pump attachment set adapters 26, 26′, 26″ together with coded attachment sets 24, 24′, 24″. The attachment set adapters 26, 26′, 26″ are interchangeable, but once the infusion pump 22 is fitted with one of the attachment set adapters 26, 26′, 26″, it can only receive the corresponding coded attachment set 24, 24′, 24″. It will be appreciated that while three pairs of attachment sets and attachment set adapters are described herein, any number of pairs would be appropriate so long as each pair was unique from the others.

With reference now to all of the drawings, it is incumbent upon the attending health care provider or other authorized user (“user”) to properly provide and introduce an appropriate infusion set 30 to a patient. For instance, if the patient is to receive medicament through an intravenous route of delivery, the user would introduce an infusion set to the patient by inserting the attached small needle or cannula of the infusion set into the subcutaneous tissue of the patient. The inlet port to the infusion set would be a connector designated by ISO 80369, Small-Bore Healthcare Connectors, for use only in intravenous applications and would be connectable only to a designated, complimentary connector 84, 84′, 84″ of an attachment set 24, 24′, 24″.

The user would next select an infusion pump 22 fitted with an appropriate pump attachment set adapter 26. That is to say, of the several varieties of attachment set adapters 26, 26′, 26″, one variety would be designated for use in intravenous applications. Once installed on the pump, the designated electrical contact 64 positioned in the designated electrical contact receiving pad 62a, 62b, 62c of the adapter would connect with the corresponding detection pin 52a, 52b, 52c of the infusion pump 22, providing a signal to the pump 22 that it is to operate only within parameters preselected as appropriate for intravenous applications. Moreover, to aid in the recognition and identification of pump configuration and use, a set color scheme or pump shape or both can be correlated with a particular pump use. For instance, with reference to FIGS. 2a, 2b, 2c, each of attachment sets 26, 26′, 26″, configured for enteral, IV, or neuraxial uses, respectively, could be identified by a coded, unique coloring of all or a portion of the visible pump exterior. Alternatively, or additionally, each of the attachment sets 24, 24′, 24″ could have a unique shape, as is presented, for example in FIGS. 2a, 2b, 2c.

Additionally, or alternatively, the pump 22 could be equipped with a small optical camera (not shown). The unique outlet port 84, 84′, 84″ of the respective attachment set 24, 24′, 24″ could be held up to the camera for an optical identification of the unique outlet port 84, 84′, 84″, and the pump 22 could effectively program itself to match the pump functionality to the functionality associated with the outlet port 84, 84′, 84″.

As a further identification protocol, the unique outlet ports 84, 84′, 84″ of the attachment sets 24, 24′, 24″ can be fitted with keyed removable protective end caps (not shown). The pump 22 could be fitted with unique, complementary portals, for receiving the protective end caps; upon pump setup, the end cap would be removed from the outlet port 84, 84′, 84″, and inserted into a complementary pump portal, thereby signaling to the pump 22 the type of pump functionality required by and associated with the attachment set 24, 24′, 24″. The pump 22 could accordingly set up the corresponding delivery mode with appropriate drug library, display color scheme and warnings and alerts. The pump 22 could be programmed to be inoperative if more than one such connector were inserted. Additionally, the pump 22 could be programmed such that the connector would have to be removed, and a connector end cap reinserted into the portal, when the attachment set 24 is changed.

With the appropriate infusion set 30 introduced to the patient, and an infusion pump 22 selected that is properly fitted with an attachment set adapter 26 that matches the performance of the pump 22 to the selected type of infusion set, the user selects an appropriate attachment set 24, 24′, 24″. The appropriate attachment set 24, 24′, 24″ will have an outlet port 84, 84′, 84″ compatible with the inlet port of the infusion set 30. It will be recalled, from the description above and with reference to the drawings, that the outlet connector 84, 84′, 84″ is keyed to (coordinated with) the position of guide fin 108, 108′, 108″ on the pumping port 86, 86′, 86″ of the attachment set 24, 24′, 24″. It will also be recalled that the position of the fin receiving notch 74, 74′, 74″ in the notch plate 70, 70′, 70″ of the pump attachment set adapter 26, 26′, 26″ is keyed to (coordinated with) the position of the pad 62a, 62b, 62c that retains the electrical contact 64. It will accordingly be appreciated that, because the operation of the infusion pump 22 is keyed to the placement of the contact 64 on the pump attachment set adapter 26, 26′, 26″, and that the placement of the contact 64 is also coordinated with the placement of the fin receiving notch 74, 74′, 74″ on the selected pump attachment set adapter 26, 26′, or 26″, and that the placement of the guide fin 108, 108′, or 108″ of the pumping port 86 is keyed to the particular outlet connector 84, 84′, or 84″ of an attachment set, it necessarily follows that the operation of the pump 22 can be exclusively keyed to the selected type of infusion set with the installation of an appropriate attachment set 24, 24′, 24″. More particularly, with the proper coordination of the above described keyed elements, a pump can selectively be operated in a mode of operation that is exclusive to a particular type of ISO compliant connector, and that the mode of operation of the pump can be changed by, but can only be changed by, fitting the pump 22 with alternate pump attachment set adapters 26, 26′, 26″.

With reference to FIG. 10, alternative pump attachment set adapter 126 enables the multipurpose infusion pump 22 to be adapted to uniquely receive an alternatively designed attachment set 124. In particular, shiftable member 132 is selectively shiftable to a plurality of positions, wherein one or more of the positions shifts shiftable member 132 including slot 134 into a position adapted to uniquely receive guide fin 188 of pumping port 186. An indicator pin 190 on pumping port 186 pressing on at least one of detection pins 52a-c. It will be appreciated that if shiftable member 132 is shifted to another of the one or more positions, the pump attachment set adapter 126 can be adapted to uniquely receive guide fin (not shown) of a different pumping port (not shown). In particular, the guide fin of the different pumping port can be oriented in a different lateral position, with the lateral position of the fin of the different particular pumping port being correlating to a different route of delivery.

With reference to FIG. 10, alternative pump attachment set adapter 126 enables the multipurpose infusion pump 22 to be adapted to uniquely receive an alternatively designed attachment set 124. In particular, shiftable member 132 is selectively shiftable to a plurality of positions, wherein one or more of the positions shifts shiftable member 132 including slot 134 into a position adapted to uniquely receive guide fin 188 of pumping port 186. An indicator pin 190 on pumping port 186 pressing on at least one of detection pins 52a-c. It will be appreciated that if shiftable member 132 is shifted to another of the one or more positions, the pump attachment set adapter 126 can be adapted to uniquely receive guide fin (not shown) of a different pumping port (not shown). In particular, the guide fin of the different pumping port can be oriented in a different lateral position, with the lateral position of the fin of the different particular pumping port being correlating to a different route of delivery. Control over the position of key 130 can be done either administratively or mechanically (e.g., by virtue of a particular position of guide fin 188 of correspondingly particular pumping port 186) or both, as is need to exert proper control over the assignment of pump 22 operation.

In an embodiment, a method of identifying a particular infusion route to an infusion pump, the infusion pump including a pump inlet and a pump outlet, comprises providing an attachment set operably, removably couplable to the pump inlet and the pump outlet, the attachment set comprising (i) an attachment set inlet connector operably, detachably coupling the attachment set to a medicament container, (ii) a coded attachment set outlet connector operably, detachably coupling the attachment set to an infusion set, the coded attachment set outlet connector being of a particular type selected from a group of different types of ISO 80369 compliant connectors and thereby identifying a particular infusion route, and (iii) a coded attachment set pump fitting in fluid communication with the attachment set inlet connector and the coded attachment set outlet connector, the coded attachment set pump fitting being operably, communicatively couplable with the pump to convey to the pump information regarding the coded attachment set outlet connector, and thereby identifying to the pump the particular infusion route.

In embodiments, the method can further comprise providing a pump attachment set adapter operably, removably coupled to the pump for removably receiving the coded attachment set pump fitting, the pump attachment set adapter configured to operably, exclusively receive only a particular type of coded attachment set pump fitting selected from a plurality of differently coded types of attachment set pump fittings, whereby the operation of the pump is responsive to and exclusive to the coded attachment set outlet connector.

In embodiments, the method can further comprise programming the pump such that operation of the pump is responsive and exclusive to the coded attachment set outlet connector.

In embodiments, the method can further comprise programming the pump with the information regarding the coded attachment set outlet connector to enable the pump to identify the particular infusion route.

Programming a pump can comprise uploading or downloading data or information to or from a pump; providing, inserting or coupling a module, memory or other device to a pump; entering an instruction or information into a pump; accepting an instruction or information by a pump; or any other way of transferring data information to or from a pump utilizing hardware, software, firmware, wired communications, wireless communications and/or other devices or methodologies. Example devices that can be used to program a pump can include one or more of a computing device, a server, a pump programming device, a cloud device, a host device, an engine, a handheld device, a telephonic device, a dedicated programming device, and/or other devices.

In an embodiment, an attachment set removably couplable to an infusion pump comprises a coded attachment set outlet connector configured to couple the attachment set to an infusion set; and a coded attachment set pump fitting configured to couple the attachment set to the infusion pump and thereby identify to the pump a particular infusion route.

Regardless of a particular embodiment of subject matter hereof, it is to be appreciated and understood that, in general, any suitable alternatives may be employed to provide novel and inventive medicament infusion systems and pump assemblies as described by example or otherwise contemplated herein. It is also to be appreciated and understood that compositions, sizes, and strengths of various components described herein are all a matter of design choice depending upon intended uses thereof. Accordingly, these and other various changes or modifications in form and detail may also be made, without departing from the true spirit and scope of novel and inventive medicament infusion systems and pump assemblies defined by the appended claims.

1. A medicament infusion system for delivering a medicament, the medicament delivered from a medicament container, through an infusion set, to a patient, via an infusion route selected from one of a plurality of infusion routes, comprising:
a pump having a pump inlet and a pump outlet; and
an attachment set operably, removably couplable to the pump inlet and the pump outlet, including—
an attachment set inlet connector operably, detachably coupling the attachment set to the medicament container,
a coded attachment set outlet connector operably, detachably coupling the attachment set to the infusion set, the coded attachment set outlet connector being of a particular type selected from a group of different types of ISO 80369 compliant connectors, the coded attachment set outlet connector thereby identifying a particular infusion route, and
a coded attachment set pump fitting in fluid communication with the attachment set inlet connector and the coded attachment set outlet connector, the coded attachment set pump fitting being operably, communicatively couplable with the pump to convey to the pump information regarding the coded attachment set outlet connector, and thereby identifying to the pump the particular infusion route.

2. The medical infusion system claimed in claim 1, further comprising a pump attachment set adapter operably, removably coupled to the pump for removably receiving the coded attachment set pump fitting, the pump attachment set adapter configured to operably, exclusively receive only a particular type of coded attachment set pump fitting selected from a plurality of differently coded types of attachment set pump fittings, whereby the operation of the pump is responsive to and exclusive to the coded attachment set outlet connector.

3. A method of identifying a particular infusion route to an infusion pump, the infusion pump including a pump inlet and a pump outlet, the method comprising:
providing an attachment set operably, removably couplable to the pump inlet and the pump outlet, the attachment set comprising (i) an attachment set inlet connector operably, detachably coupling the attachment set to a medicament container, (ii) a coded attachment set outlet connector operably, detachably coupling the attachment set to an infusion set, the coded attachment set outlet connector being of a particular type selected from a group of different types of ISO 80369 compliant connectors and thereby identifying a particular infusion route, and (iii) a coded attachment set pump fitting in fluid communication with the attachment set inlet connector and the coded attachment set outlet connector, the coded attachment set pump fitting being operably, communicatively couplable with the pump to convey to the pump information regarding the coded attachment set outlet connector, and thereby identifying to the pump the particular infusion route.

4. The method of claim 3, further comprising providing a pump attachment set adapter operably, removably coupled to the pump for removably receiving the coded attachment set pump fitting, the pump attachment set adapter configured to operably, exclusively receive only a particular type of coded attachment set pump fitting selected from a plurality of differently coded types of attachment set pump fittings, whereby the operation of the pump is responsive to and exclusive to the coded attachment set outlet connector.

5. The method of claim 4, further comprising programming the pump such that operation of the pump is responsive and exclusive to the coded attachment set outlet connector.

6. The method of claim 4, further comprising operably coupling the pump attachment set adapter to the pump.

7. The method of claim 3, further comprising programming the pump with the information regarding the coded attachment set outlet connector.

8. The method of claim 3, further comprising operably coupling the attachment set to the pump inlet and the pump outlet.

9. An attachment set removably couplable to an infusion pump, comprising:
a coded attachment set outlet connector configured to couple the attachment set to an infusion set; and
a coded attachment set pump fitting configured to couple the attachment set to the infusion pump and thereby identify to the pump a particular infusion route.
